(19) United States
(12) Patent Application Publication
(10) Pub. No.: US 2006/0122432 A1
(43) Pub. Date: Jun. 8, 2006

(54) Aryl piperidine derivatives and use thereof to reduce elevated levels of ldl-cholesterol

(76) Inventors: Anne Marie Jeanne Bouillot, Les Ulis (FR); Bernard Andre Dumaitre, Les Ulis (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/520,754

(22) PCT Filed: Jul. 11, 2003
(86) PCT No.: PCT/EP03/07615

§ 371 Date: Jan. 10, 2005

(30) Foreign Application Priority Data
Jul. 12, 2002 (GB) 02162337

(51) Int. Cl. C07C 211/00; A61K 9/00

(52) U.S. Cl. 564/463; 424/400

(57) ABSTRACT

This invention relates to novel compounds which up-regulate LDL receptor (LDL-r) expression and to processes for their preparation, pharmaceutical compositions containing them and their medical use. More particularly, this invention relates to novel aromatic piperidines and their use in therapy.

This invention relates to novel compounds which up-regulate LDL receptor (LDL-r) expression and to processes for their preparation, pharmaceutical compositions containing them and their medical use. More particularly, this invention relates to novel aromatic piperidines and their use in therapy.

Epidemiological studies have clearly demonstrated the correlation between reduction in plasmatic LDL cholesterol and the benefit on cardiovascular events including mortality. LDL cholesterol is eliminated from plasma by specific binding to LDL-r expressed by the liver. Regulation of LDL-r expression occurs in the liver and is mainly dependent on intracellular cholesterol concentration. Increasing free cholesterol concentration leads to a reduced LDL-r expression through a mechanism involving transcriptional factors. Counteracting with this process is expected to up-regulate LDL-r expression in the liver and to increase the clearance of LDL cholesterol.

International Patent Application Number PCT/EP00/06668 concerns the novel use of the SREBP-cleavage activating protein (SCAP) in a screening method. Two compounds are disclosed, namely 4-(4-chloro-benzoylamino)-N-4-[4-(2-ethoxy-4-ethyl-phenyl)-piperidin-1-yl]-butyl)benzamide and 4-(4-benzoyl)-N-4-[4-(4-isopropyl-2-methoxy-phenyl)-piperidin-1-yl]-butyl)benzamide hydrochloride, which do not form part of the present invention.

Another publication, Bioorganic and Medicinal Chemistry Letters Vol. 5, 3, 219-222, 1995 discloses compounds having the general formula (A) 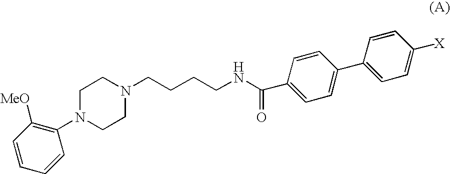
where X may be COMe, SO₂Me and NH₂, as having high affinity for the dopamine D₃ receptor and postulates their use in CNS disorders, particularly psychiatric illness. The compound of formula A where X is COMe is also disclosed in J. Pharmacol. Exp. Ther. 287; 1 1998 187-197 and Bioorganic and Medicinal Chemistry Letters Vol. 7, 15, 1995-1998, 1997, again as being useful in treating CNS disorders. It will be noted that the present invention differs from the compounds of formula (A) in use of a piperidine ring rather than a piperazine and in the utility disclosed.

Journal Of Medicinal Chemistry, Vol. 40, 6, 952-960,1997 discloses compounds of formula (B) 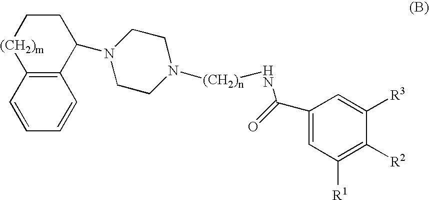
where m=0, 1 or 2; n=2 or 3; R¹ and R³=H or OMe and R² may be Ph, as selective 5-HT_{1A} receptor ligands having CNS activity. It will be noted that the examples of the present invention differ from those of formula (B) in use of a piperidine ring rather than a piperazine and in the utility disclosed.

International Patent Application Publication Number WO99/45925 discloses compounds of formula (C) 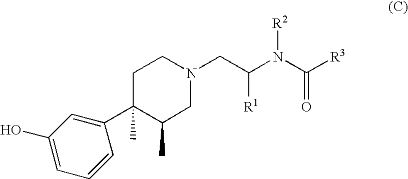
where R¹ may be hydrogen, R² may be hydrogen and R³ may be a group 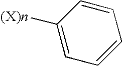
where X may be an aryl group and n may be 1. Specifically disclosed are compounds where the group COR³ is formed from 2- and 4-biphenyl carboxylic acid and R¹ and R² are methyl or hydrogen respectively. The utility of the compounds is as opioid receptor binding agents which may be useful as analgesics. The substitution on the 3- and 4-positions of the piperidine ring leave the compounds of this publication outside the scope of the present invention. Furthermore, the utility disclosed is different.

International Patent Application Publication Number WO98/37893 discloses compounds of formula (D) 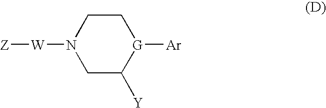
where Ar may represent an optionally substituted phenyl or naphthyl, G may be N or CH₂ (sic), W may be an optionally substituted alkylene, Y may be hydrogen and Z may represent a group R⁴CONRr, where R⁴ may be an optionally substituted phenyl and R₅ may be hydrogen. These compounds are described as being D2 receptor antagonists useful in the treatment of CNS disorders such as Parkinson's Disease. None of the compounds specifically disclosed fall within the scope of the present invention and the disclosed utlity is different.

International Patent Application Publication Number WO9402473 discloses compounds of formula (E)

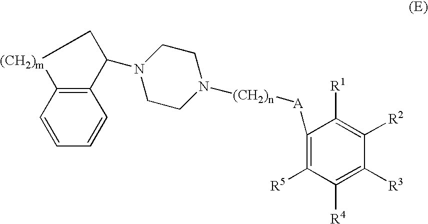

where A is —NHCO— or —CONH—; R₁-R₅ may be hydrogen or phenyl, m may be 1-3 and n may be 1-3. Specifically disclosed are the following compounds:

| No. | A | n | m | R¹ | R² | R³ | R⁴ | R⁵ |
|---|---|---|---|---|---|---|---|---|
| 5 | NHCO | 2 | 1 | H | H | Ph | H | H |
| 12 | NHCO | 2 | 2 | H | H | Ph | H | H |
| 19 | NHCO | 2 | 3 | H | H | Ph | H | H |

The compounds are described as 5HT-1A agonists having CNS activity and may be used as anti-depressants, anti-hypertensive, analgesics etc. It will be noted that the examples of the present invention differ from those of formula (E) in use of a piperidine ring rather than a piperazine and in the utility disclosed.

International Patent Application Publication Number WO99/45925 discloses compounds of formula (F) 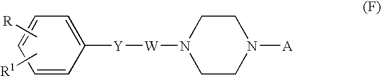
where A may represent a substituted phenyl group, W represents a linear or branched alkylene group having from 2 to 6 carbon atoms; Y may represent a group NHCO or CONH; and R may be a substituted phenyl group. Particularly disclosed is the compound G

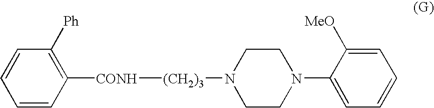

These compounds are described as being α1A-adrenergic receptors useful in the treatment of contractions of the prostate, urethra and lower urinary tract, without affecting blood pressure. It will be noted that the examples of the present invention differ from those of formula (G) in use of a piperidine ring rather than a piperazine and in the utility disclosed.

International Patent Application Publication Number WO98/35957 describes compounds of formula (H) 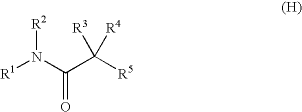
wherein R¹-R⁵ are each individually selected from the group of substituents including hydrogen, halogen, hydroxyl, thiol, lower alkyl, substituted lower alkyl, alkenyl, alkynyl, alkylalkenyl, alkylalkynyl, alkoxy, alkylthio, acyl, aryloxy, amino, amido, carboxyl, aryl, substituted aryl, heterocycle, heteroaryl, substituted heterocycle, heteroalkyl, cycloalkyl, substituted cycloalkyl, alkylcycloalkyl, alkylcycloheteroalkyl, nitro and cyano. Specifically disclosed compounds are those formed by the N-alkylation of a a substituted piperidine or piperazine with a group (J) 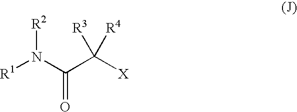
where X is a leaving group. None of the compounds specifically disclosed fall within the scope of the present invention and the invention is in no way suggested by the disclosure. The compounds are said to be of use as NPY Y5 receptor antagonists in the treatment of obesity, bulemia and related disorders and NPY Y5 receptor inhibition related disorders such as memory disorders, epilepsy, dyslipidemia and depression.

Journal Of Medicinal Chemistry, Vol. 31, 1968-1971, 1988 discloses certain aryl piperazines compounds, which fall outside the present invention, as 5HT-1a Serotonin Ligands as potential CNS agents. Specifically disclosed are compounds of formula (K) 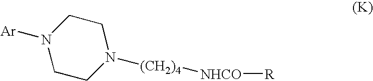
where Ar=Ph and R=Ph, Ar=2-methoxyphenyl and R=Ph and Ar=2-pyrimidyl and R=Ph.

Journal Of Medicinal Chemistry, Vol. 34, 2633-2638, 1991 discloses aryl piperazines having reduced al adrenergic affinity. Specifically disclosed is the compound (L) 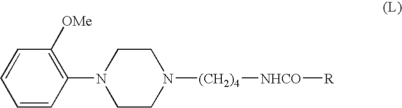
where R is 4-(BnO)-phenyl, which falls outside the scope of the present invention.

The present invention provides aryl piperidine derivatives which are particularly useful in treating cardiovascular disorders associated with elevated levels of circulating LDL-cholesterol.

According to a first aspect, the invention provides a compound of formula (I) 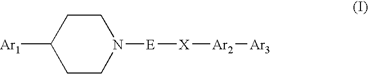
wherein
Ar₁ is:
(i) phenyl, naphthyl or phenyl fused by a C₃₋₈ cycloalkyl; or
(ii) heterocyclyl selected from the list consisting of: monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated or aromatic, provided that at least one ring is aromatic;
   where Ar₁ is independently substituted by at least one R¹ group and is independently substituted by 0-3 R³ groups;
Ar₂ is a phenyl group, a 5-6 membered heteroaromatic group or a bicyclic heteroaromatic group, each of which are optionally substituted by 1-4 groups independently selected from the list: C₁₋₄-allyl, halogen, hydroxy, C₁-alkoxy, C₁₋₆-acyl, C₁₋₆-acyloxy, amino, C₁₋₄alkylamino, di-C₁₋₄alkylamino, —(CH₂)ₙOH, —(CH₂)ₙNRₓR_{y}, —O(CH₂)ₙO(CH₂)ₘOR², —O(CH₂)ₙC(O)NRₓR_{y}, —O(CH₂)ₙCN, C₂ alkenyl, —O(CH₂)ₙCO₂R², —OSO₂(CH₂)ₚCH₃, —OSO₂NRₓR_{y} and —CO₂(CH₂)ₚCH₃;
Ar₃ is:
(i) phenyl, naphthyl or phenyl fused by a C₃₋₈cycloalkyl; or
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic;
   wherein Ar₃ is optionally substituted by 1-4 groups independently selected from the group consisting of: hydroxy, C₁₋₄alkyl, C₁₋₄alkoxy, C₂₋₄alkenyl, C₂₋₄alkenyloxy, C₁₋₄perfluoroalkoxy, C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, nitro, C₁₋₄perfluoroalkyl, C₁₋₄alkylcarbonyl, fluoroC₁₋₄alkylcarbonyl, C₁₋₄alkoxycarbonyl, aminocarbonyl, C₁₋₄alkylaminocarbonyl, di-C₁₋₄alkylaminocarbonyl, C₁₋₄alkylsulfonyl, C₁₋₄alkylaminosulfonyl, di-C₁₋₄alkylaminosulfonyl, C₁₋₄alkylsulfonyl and C₁₋₄alkylsulfoxy;
E is —C₁₋₆alkylene-;
X is —CONR²— or —NR²CO—;
   wherein
R¹ is O(CH₂)ₙOR²;
R² is C₁₋₄alkyl or hydrogen;
R³ is halogen, C₁₋₄alkoxy or C₁₋₄alkyl;
Rₓ and R_{y} are independently C₁₋₄alkyl or hydrogen;
n and m are independently 1-4; and
p is 0-4;
   or a physiologically acceptable prodrug, salt or solvate thereof.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), alkenyl includes both straight and branched chain saturated hydrocarbon groups containing one double bond. Examples of alkenyl groups include ethenyl or n-propenyl groups.

Referring to the general formula (I), acyl refers to aliphatic or cyclic hydrocarbons attached to a carbonyl group through which the substituent bonds, such as acetyl.

Referring to the general formula (I), phenyl fused by a C₃₋₈cycloalkyl includes bicyclic rings such as 1,2,3,4-tetrahydronaphthyl, which, for the avoidance of doubt, is linked to the rest of the molecule through the aromatic ring.

Referring to general formula (I), a halogen atom includes fluorine, chlorine, bromine or iodine.

Referring to the general formula (I), C₁₋₃-perfluoroalkyl and C₁₋₃perfluoroalkoxy includes compounds in which the hydrogens have been partially or fully replaced by fluorines, such as trifluoromethyl and trifluoromethoxy or trifluoroethyl.

Referring to the general formula (I), a 5-6 membered heteroaromatic group includes a single aromatic ring system containing at least one ring heteroatom examples include pyridyl and thiazolyl.

Referring to the general formula (I), a 3-7 membered heterocyclyl group means any single ring system containing at least one ring heteroatom independently selected from O, N and S, wherein said ring is saturated, unsaturated or aromatic.

Preferably Ar₁ is phenyl, naphthyl, 1,2,3,4-tetrahydronaphthyl, indolyl, benzofuranyl, benzothiophenyl or indazolyl. More preferably Are is phenyl or 1,2,3,4-tetrahydronaphthyl. Most preferably Are is 1,2,3,4-tetrahydronaphthyl.

Where Ar₁ is 1,2,3,4-tetrahydronaphthyl, the link to the piperidine ring is preferably through the 2-position of the 1,2,3,4-tetrahydronaphthyl moiety and mono-substitution by R¹ is in the corresponding 1-position.

Preferably E is n-butylene.

Preferably X is —NR²CO—. Preferably R² is hydrogen.

Preferably Ar₂ is phenyl or a 5-6-membered heteroaromatic group. More preferably Ar₂ is phenyl, pyridyl, thiazolyl, oxazolyl or imidazolyl. More preferably Ar₂ is phenyl, oxazolyl or thiazolyl.

Preferably Ar₃ is phenyl, pyridyl or thienyl, more preferably phenyl. Preferably Ar₃ is substituted by halogen (e.g. chloro), C₁₋₄perfluoroalkyl (e.g. trifluoromethyl), nitrile, C₁₋₄acyl (e.g. acetyl), C₁₋₄alkylsulfonyl (e.g. methylsulfonyl) or C₁₋₄alkylsulfonylamino.

When Ar₃ is phenyl, para-substitution is preferred.

Preferably R¹ is OCH₂CH₂OR².

Preferably Ar₁ is not substituted by R³, however when Ar₁ is substituted by R³ preferred substituents are C₁₋₄alkyl or C₁₋₄alkoxy.

Preferably Ar₂ is optionally substituted by C₁₋₄alkyl, halogen, hydroxy, hydroxyC₁₋₄alkyl or C₁₋₄alkoxy. Preferably Ar₂ is optionally substituted by C₁₋₄alkyl or hydroxyC₁₋₄alkyl.

Particularly preferred compounds of the invention include those in which each variable in Formula (I) is selected from the preferred groups for each variable. Even more preferable compounds of the invention include those where each variable in Formula (I) is selected from the more preferred or most preferred groups for each variable.

Preferably
Ar₁ is phenyl, naphthyl, 1,2,3,4-tetrahydronaphthyl, indolyl, benzofuranyl, benzothiophenyl or indazolyl; where Ar₁ is independently substituted by at least one R¹ group and is independently substituted by 0-3 R³ groups;
Ar₂ is phenyl, pyridyl, thiazolyl, oxazolyl or imidazolyl, each of which are optionally substituted by 14 groups independently selected from the list; C₁₋₄alkyl, halogen, hydroxy, hydroxyC₁₋₄alkyl and C₁₋₄alkoxy;
Ar₃ is phenyl, pyridyl or thienyl, wherein Ar₃ is optionally substituted by 1-4 groups independently selected from the group consisting of: halogen (e.g. chloro), C₁₋₄perfluoroalkyl (e.g. trifluoromethyl), nitrile, C₁₋₄acyl (e.g. acetyl), C₁₋₄alkylsulfonyl (e.g. methylsulfonyl) and C₁₋₄alkylsulfonylamino;
E is n-butylene;
X is —NR²CO—;
   wherein
R¹ is —OCH₂CH₂OR²;
R² is C₁₋₄alkyl or hydrogen;
R³ is halogen, C₁₋₄alkoxy or C₁₋₄alkyl; and
n is 1-4.

Preferred compounds of formula (I) are selected from the list:
4-Hydroxymethyl-2-(4-trifluoromethyl-phenyl)-thiazole-5-carboxylic acid (4-{4-[1-(2-hydroxy-ethoxy)-5,6,7,8-tetrahydro-naphthalen-2-yl]-piperidin-1-yl}-butyl)-amide (Example 3);
4′-Cyano-biphenyl-4-carboxylic acid (4-{4-[1-(2-hydroxy-ethoxy)-5,6,7,8-tetrahydro-naphthalen-2-yl]-piperidin-1-yl)butyl)-amide hydrochloride (Example 5);
2-(4-Cyano-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide, hydrochloride (Example 7);
2-(2,4-dichloro-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide (Example 8);
2′,4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide (Example 9);
4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide (Example 10); and
4′-Methanesulfonylamino-biphenyl-4-carboxylic acid (4-{4-[1-(2-hydroxy-ethoxy)-5,6,7,8-tetrahydro-naphthalen-2-yl]-piperidin-1-yl}-butyl)-amide (Example 12).

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

As used herein the term ‘physiologically acceptable’ means a compound which is suitable for pharmaceutical use.

Suitable physiologically acceptable salts of the compounds of general formula (I) include acid addition salts formed with pharmaceutically acceptable inorganic acids for example, phosphates, hydrochlorides, hydrobromides or sulphates, or with pharmaceutically acceptable organic acids for example mesylates, lactates and acetates. More suitably, a physiologically acceptable salt of the compounds of general formula (I) is a phosphate salt.

The solvates may, for example, be hydrates.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like.

Hereinafter, compounds, their pharmaceutically acceptable salts, their solvates and polymorphs, defined in any aspect of the invention (except intermediate compounds in chemical processes) are referred to as “compounds of the invention”.

Compounds of the invention may be administered as the raw chemical but the active ingredient is preferably presented as a pharmaceutical formulation.

Compounds of the invention may be formulated for oral, buccal, parenteral, transdermal, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or lozenges formulated in conventional manner.

For transdermal administration the compounds of the invention may be formulated as creams, gels, ointments or lotions or as a transdermal patch. Such compositions may for example be formulated with an aqueous or oily base with the addition of suitable thickening, gelling, emulsifying, stabilising, dispersing, suspending, and/or colouring agents.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unit dose device or alternatively as a powder mix with a suitable carrier for administration using a suitable delivery device.

The compositions may contain from 0.1% upwards, e.g. 0.1-99% of the active material, depending on the method of administration. A proposed dose of the compounds of the invention is 0.25 mg/kg to about 125 mg/kg bodyweight per day e.g. 20 mg/kg to 100 mg/kg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

The compounds of the invention may, if desired, be administered with one or more therapeutic agents and formulated for administration by any convenient route in a conventional manner. Appropriate doses will be readily appreciated by those skilled in the art. For example, the compounds of the invention may be administered in combination with an HMG CoA reductase inhibitor, an agent for inhibition of bile acid transport or fibrates.

The compounds of the invention are inducers of LDL-r expression and are thus of use in the treatment of conditions resulting from elevated circulating levels of LDL-cholesterol. Thus compounds of the invention are of use in the treatment of diseases in which lipid imbalance is important, e.g. atherosclerosis, pancreatitis, non-insulin dependent diabetes mellitus (NIDDM), coronary heart diseases and obesity. In addition compounds of the invention are also useful in lowering serum lipid levels, cholesterol and/or triglycerides, and are of use in the treatment of hyperlipemia, hyperlipidemia, hyperlipoproteinemia, hypercholesterolemia and/or hypertriglyceridemia.

It will be appreciated that reference to treatment is intended to include prophylaxis as well as the alleviation of established symptoms.

Compounds of the invention may be prepared in a variety of ways. In the following reaction schemes and hereafter, unless otherwise stated groups Ar₁, Ar₂, Ar₃, R¹, R², R³, R⁴, E and X are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of formula (Ia), i.e. compounds of formula (I) where X is —NR²C(O)— where the nitrogen is attached to E, may be prepared according to reaction scheme 1 by reacting compounds of formula (II) with compounds of formula (III) where L is a leaving group such as halogen or hydroxy, using standard amide coupling conditions detailed in the experimental section.

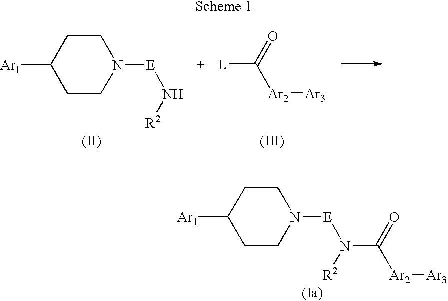

Compounds of formula (Ib), i.e. compounds of formula (I) where R¹ is —OR, may be prepared from the corresponding hydroxy compound (IV) according to reaction scheme 2. Preferred reaction conditions comprise treating (IV) with a suitable base such as sodium hydride or caesium carbonate followed by addition of RL where L is a leaving group such as halogen. Compounds of formula (IV) may be prepared by adapting methods described herein for the preparation of compounds of formula (I).

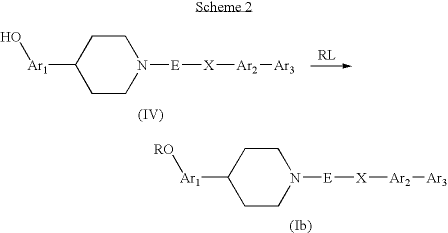

Compounds of formula (I) where Ar₁ is a nitrogen containing heterocycle may be substituted on the nitrogen by nucleophilic substitution. For instance where Ar₁ is indol-3-yl, substitution may be effected by treating (I) with base such as sodium hydride followed by reaction with a suitable nucleophile.

Compounds of formula (I) may be prepared be coupling boronic acid compounds of formula (V) with compounds of formula (VI) according to reaction scheme 3. Preferred reaction conditions comprise treatment with Pd(PPh₃)₄ and a suitable base such as sodium carbonate at elevated temperature.

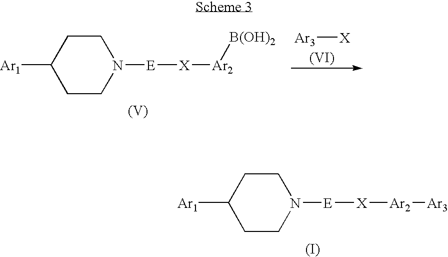

Compounds of formula (Ic) may be prepared by removal of protection group P frrom compounds of formula (XI) according to reaction scheme 4. A preferred protecting group P is tetrahydropyran-2-yl which may be be removed by acid hydrolysis, typicially reaction with dilute hydrochloric acid in methanol at room temperature.

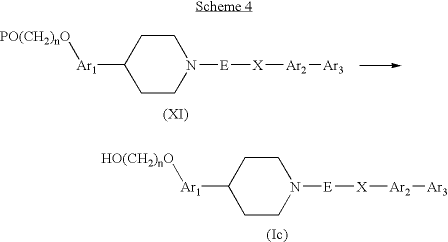

Compounds of formula (XI) may be prepared by adapting methods described herein for the preparation of compounds of formula (I).

Compounds of formula (II) may be prepared according to reaction scheme 5 by reacting a compound of formula (VII) with a compound of formula (VIII) where L is a leaving group such as halogen and P is a suitable protecting group. Preferred conditions comprise reaction with a suitable base such as potassium carbonate. Removal of protecting group P gives compounds of formula (II). A preferred nitrogen protecting group is where the nitrogen attached to E and group R² form phthalimide (i.e. 1,3-dioxo-1,3-dihydro-isoindol-2-yl). Removal of the phthalimide protecting group gives compounds of formula (II) where R² is hydrogen. Preferred conditions comprise treatment with hydrazine at elevated temperature.

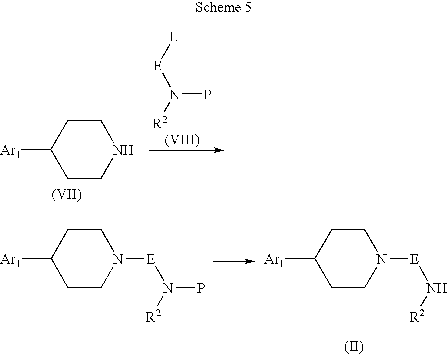

Compounds of formula (VII) may be prepared by methods described in the experimental section hereinbelow. Compounds of formula (VIII) are either known or may be prepared from known compounds by methods available to the skilled person.

Compounds of formula (IIIa), i.e. compounds of formula (III) (see reaction scheme 1) where L is hydroxy, may be prepared according to reaction scheme 5 by coupling boronic acid compounds of formula (IX) with compounds of formula (X) where L is a leaving group such as halogen under analogous conditions described for reaction scheme 3.

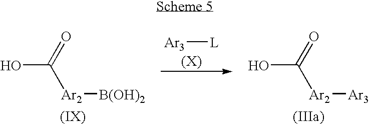

Compounds of formula (IX) and (X) are either known or may be prepared from known compounds by methods available to the skilled person.

The protecting groups used in the preparation of compounds of formula (I) may be used in conventional manner. See for example ‘Protective Groups in Organic Chemistry’ Ed. J. F. W. McOmie (Plenum Press 1973) or ‘Protective Groups in Organic Synthesis’ by Theodora W Greene and P M G Wuts (John Wiley and Sons 1991). Conventional amino protecting groups may include for example aralkyl groups, such as benzyl, diphenylmethyl or triphenylmethyl groups; and acyl groups such as N-benzyloxycarbonyl or t-butoxycarbonyl. Conventional carboxylic acid protecting groups include methyl and ethyl groups.

It will be appreciated that the invention includes the following further aspects. The preferred embodiments described for the first aspect extend these further aspects:

i) a pharmaceutical composition comprising a compound of the invention and a pharmaceutically acceptable carrier or diluent;

ii) the use of a compound of the invention in the manufacture of a medicament for use in the treatment of conditions resulting from elevated circulating levels of LDL-cholesterol;

iii) the use of a compound of the invention in the manufacture of a medicament for the treatment or prophylaxis of a disorder in which lipid imbalance is important (such as atherosclerosis, pancreatitis, non-insulin dependent diabetes mellitus (NIDDM), coronary heart diseases and obesity);

iv) the use of a compound of the invention in the manufacture of a medicament for lowering serum lipid levels, cholesterol and/or triglycerides;

v) the use of a compound of the invention in the manufacture of a medicament for the treatment or prophylaxis of hyperlipemia, hyperlipidemia, hyperlipoproteinemia, hypercholesterolemia and/or hypertriglyceridemia;

vi) a compound of the invention for use as a medicament;

vii) a method of treatment or prophylaxis of a disorder resulting from elevated circulating levels of LDL-cholesterol in a human patient comprising administering to the human an effective amount of a compound of the invention;

viii) a method of lowering serum lipid levels, cholesterol and/or triglycerides in a human patient comprising administering to the human an effective amount of a compound of the invention; and

ix) a combination of a compound of the invention with an HMG CoA reductase inhibitor, an agent for inhibition of bile acid transport or a fibrate.

According to a further aspect, the invention provides a compound of formula (I) 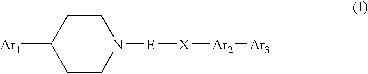
wherein
Ar₁ represents
   (i) phenyl, naphthyl, or phenyl fused by a C₃₋₈cycloalkyl, or
   (ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, provided that at least one ring is aromatic,
      where Ar₁ bears at least one group independently represented by R¹ and 0-3 groups independently represented by R³;
R¹ is O(CH₂)ₙOR²;
R² is H or (CH₂)ₘCH₃;
n is 1-4;
m is 0-4;
R³ is selected from halogen, —O—(C₀₋₄ alkylene)-R⁴ or —(C₀₋₄alkylene)-R⁴, where each alkylene group may additionally incorporate an oxygen in the chain, with the proviso that there are at least two carbon atoms between any chain heteroatoms;
R⁴ represents
   (i) hydrogen, C₁₋₄ perfluoroalkyl, C₁₋₄perfluoroalkoxy,
   (ii) phenyl, phenyl fused by a C₃₋₈ cycloalkyl, naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,
   (iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-CIA alkylamino, or
   (iv) amino, C₁₋₄ alkylamino or di-C₁₋₄alkylamino;
Ar₂ represents phenyl or a 5-6 membered heteroaromatic group or a bicyclic heteroaromatic group, where each group is optionally substituted by one or two groups independently selected from the group consisting of: C₁₋₄ alkyl, halogen, hydroxy, C₁₋₄ alkoxy, C₁ acyl, C₁₋₆ acyloxy, amino, C₁₋₄ alkylamino and di-C₁₋₄ alkylamino groups;
Ar₃ represents
   (i) phenyl, naphthyl, or phenyl fused by a C₃₋₈cycloalkyl,
   (ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
where Ar₃ is optionally substituted by 1-4 groups independently selected from the group consisting of: hydroxy, alkyl, C₁₋₄ alkoxy, C₂₋₄ alkenyl, C₂₋₄ alkenyloxy, C₁₋₄perfluoroalkoxy, C₁₋₄ acylamino or an electron withdrawing group selected from the list consisting of: nitrile, nitro, C₁₋₄, C₁₋₄ perfluoroalkyl, C₁₋₄ acyl, C₁₋₄ alkoxycarbonyl, aminocarbonyl, C₁₋₄alkylaminocarbonyl; di-C₁₋₄ alkylaminocarbonyl, C₁₋₄ alkylsulfonyl, C₁₋₄ alkylaminosulfonyl and di-C₁₋₄ alkylaminosulfonyl, C₁₋₄ alkylsulfonyl and C₁₋₄alkylsulfoxy;
E represents —C₁₋₆ alkylene-;
X represents —CON(H or C₁₋₄alkyl)- or —N(H or C₁₋₄alkyl)CO—; or a physiologically acceptable prodrug, salt or solvate thereof.

The invention is further described with reference to the following non-limiting examples.

Abbreviations:

Pd(PPh₃)₄—Tetrakis-(triphenylphosphine)-palladium(0), THF—Tetrahydrofuran, BF₃-Et₂O—Boron trifluoride diethyl etherate, DCM—Dichloromethane, TEA—triethylamine, CH₃CN—Acetonitrile, EtOH—Ethanol, EtOAc—Ethyl acetate, iPr₂O—Di-isopropyl ether, iPrOH—Isopropanol, Pd/C—Palladium on carbon, Et₂O— diethyl ether, Chex—cyclohexane, MeOH—Methanol, DMF—Dimethyl formamide, DME—Ethylene glycol dimethyl ether, EDCl—1-(3-dimethylaminopropyl)-, ethylcarbodiimide hydrochloride, HOBt—1-Hydroxybenzotriazole, rt—Room temperature, AcOH—Acetic acid, NaOH—Sodium hydroxide, KOH—potassium hydroxide, LiOH, H₂O—lithium hydroxide monohydrate, HCl—Hydrochloric acid, AcOH—Acetic acid, NaH—Sodium hydride, Na₂SO₄—Sodium sulfate, CCl₄—Carbon tetrachloride, AlBN—2,2′-Azobis(2-methylpropionitrile), K₂CO₃—Potassium carbonate, Na₂CO₃—Sodium carbonate, NaCl—Sodium chloride, Cs₂CO₃—Cesium carbonate, CrO₃—Chromium(VI) oxide, BBr₃—Boron tribromide, P₄S₁₀—Phosphorus sulfide.

### Intermediate 1: 1-[4-(1-Hydroxy-5,6,7,8-tetrahydro-naphthalen-2-yl)-3,6-dihydro-2H-pyridin-1-yl]-ethanone

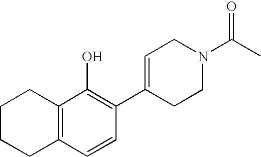

To a solution of 5,6,7,8-tetrahydro-naphthalen-1-ol (20.0 g, 0.135 mol) and 1-acetyl-4-piperidone (22.84 g, 1.2 eq.) in THF (400 mL), was added dropwise BF₃-Et₂O (68 mL, 4.0 eq). The mixture was stirred at 100° C. for 2 hours, and 14 hours at room temperature. The mixture was treated with a 1N HCl solution (400 mL). The resulting solution was extracted with DCM. The organic layer was dried over Na₂SO₄ and evaporated to dryness to give an oil which was recrystallized in acetonitrile to give the title compound (24.2 g, 89 mmol) as a white crystals in a 66% yield; GC/MS: M+C₁₇H₂₀NO₂ 271.

### Intermediate 2: 1-[4-(1-Hydroxy-5,6,7,8-tetrahydro-naphthalen-2-yl)-piperidin-1-yl]-ethanone

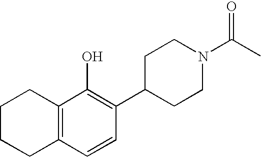

To a solution of intermediate 1 (9.4 g, 34.7 mmol) in EtOH (300 mL) was added Pd/C, 10% (0.9 g) and the reaction mixture was stirred under an atmospheric pressure of hydrogen at 25° C. for 24 hours. The mixture was filtered through a bed of celite. The filtrate was evaporated under reduced pressure to give the title compound (9.6 g, 35 mmol) as a white foam; GC/MS: M⁺C₁₇H₂₂NO₂ 273.

### Intermediate 3: 1-[4-(4-Ethyl-2-hydroxy-phenyl)-3,6-dihydro-2H-pyridin-1-yl]-ethanone

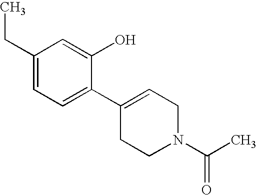

The same method was employed as in the preparation of intermediate 1 but starting from 3-ethyl-phenol and gave the title compound as a pink solid in a quantitative yield; GC/MS: M⁺ C₁₅H₁₉NO₂ 245.

### Intermediate 4: 1-[4-(4-Ethyl-2-hydroxy-phenyl)-piperidin-1-yl]-ethanone

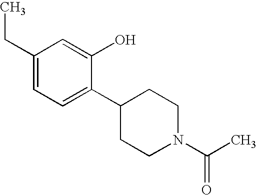

The same method was employed as in the preparation of intermediate 2 but starting from intermediate 3 and gave the title compound as a solid in a 89% yield; GC/MS: M⁺ C₁₅H₂₁NO₂ 247.

### Intermediate 5: 1-(4-{4-Ethyl-2-[2-(tetrahydro-pyran-2-yloxy)-ethoxy]-phenyl}-piperidin-1-yl)-ethanone

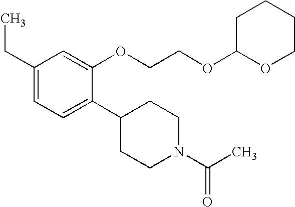

To a solution of intermediate 4 (5 g, 20 mmol) in acetone (200 mL) was added K₂CO₃ (5.52 g, 2 eq.) and 2-(2-bromo-ethoxy)-tetrahydropyran (4.58 mL, 1.5 eq.). The mixture was stirred at reflux for 48 hours and the solvent evaporated. The residue was diluted in DCM and washed with water. The organic layer was dried over Na₂SO₄ and filtered to give the title compound (11.0 g, 27 mmol) as a yellow oil in a quantitative yield after chromatography using DCM/MeOH (98/02) as eluent; GC/MS: M⁺ C₂₂H₃₃NO₄ 290 (M-Tetrahydropyran).

### Intermediate 6: 4-{4-Ethyl-2-[2-(tetrahydro-pyran-2-yloxy)-ethoxy]-phenyl}-piperidine

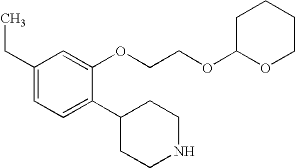

To a solution of intermediate 5 (1.0 eq) in EtOH was added a NaOH/H₂O (1/1) solution and the mixture was stirred to reflux for 24 hours. The solvent was evaporated, water added and the residue extracted with DCM. The organic layer was dried over Na₂SO₄ and the solvent evaporated to give the title compound as a brown oil; GC/MS: M⁺ C₂₀H₃₁NO₃ 333.

### Intermediate 7: 2-[4-(4-{4-Ethyl-2-[2-(tetrahydro-pyran-2-yloxy)-ethoxy]-phenyl}-piperidin-1-yl)-butyl]-isoindole-1,3-dione

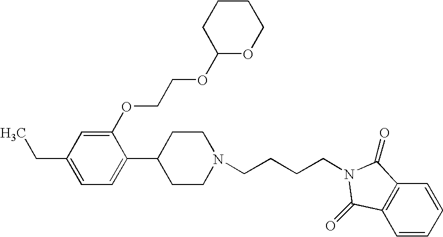

To a solution of intermediate 6 (1.0 eq) in acetone was added K₂CO₃ (2.0 eq.) and 4 bromobutyl-phthalimide (1.0 eq.) and the mixture was stirred at reflux for 6 hours. he mixture was filtered, the filtrate was evaporated and the residue was purified by flash chromatography using DCM/MeOH (95/05) as eluent to give the title compound as a brown oil in a quantitative yield; ¹H NMR (CDCl₃, 300 MHz) [symbol] 7.8 (m, 2H), 7.6 (m, 2H), 7 (d, 1H), 6.6 (d, 1H), 6.5 (s, 1H), 5.2 (s, 2H), 4.8 (m, 1H), 4.1-1.1 (m, 32H).

### Intermediate 8: 4-(4-{4-Ethyl-2-[2-(tetrahydro-pyran-2-yloxy)-ethoxy]-phenyl}-piperidin-1-yl)-butylamine

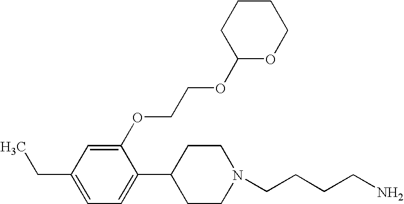

A solution of intermediate 7 (1.0 eq) in MeOH was treated with hydrazine monohydrate (2.0 eq.) and the resulting mixture was stirred to reflux for 16 hours. After cooling to rt and evaporation under reduced pressure, the residue was taken up in water and 1N HCl solution was added until pH=4. Filtration gave yellow solution that was treated with a concentrated NaOH solution. Extraction with DCM, drying over Na₂SO₄ and filtration gave the title compound as a brown oil in a 78% yield; ¹H NMR (CDCl₃, 300 MHz) δ 7.1 (d, 1H), 6.7 (d, 1H), 6.6 (s, 1H), 5.2 (s, 2H), 4.8 (m, 1H), 4.1-1.1 (m, 34H)

### Intermediate 9: 4′-Trifluoromethyl-biphenyl-4-carboxylic acid [4-(4-(4-{4-ethyl-2-[2-(tetrahydro-pyran-2-yloxy)-ethoxy]-phenyl}-piperidin-1-yl)-butyl]-amide

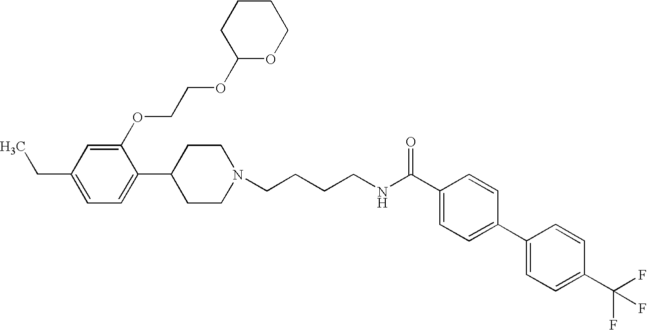

A solution of intermediate 8 (1.0 eq) in DMF was treated with 4′-trifluoromethyl-biphenyl-4-carboxylic acid (1.0 eq.), EDCl (1.5 eq.), HOBT (1.5 eq.) and TEA (1.5 eq.) and the mixture stirred for 24 hours at rt. The solvent was evaporated, and the residue was diluted with DCM and washed with water and with a 1N NaOH solution. The organic layer was dried over Na₂SO₄ and evaporated. After purification by flash chromatography using DCM/MeOH (95/5) as eluent, the title compound was obtained as white crystals in a 27% yield; LC/MS: M+H C₃₈H₄₈F₃N₂O₄ 653.

### Intermediate 10: 4′-Cyano-biphenyl-4-carboxylic acid [4-(4-{4-ethyl-2-[2-(tetrahydro-pyran-2-yloxy)-ethoxy]-phenyl}piperidin-1-yl)-butyl]-amide

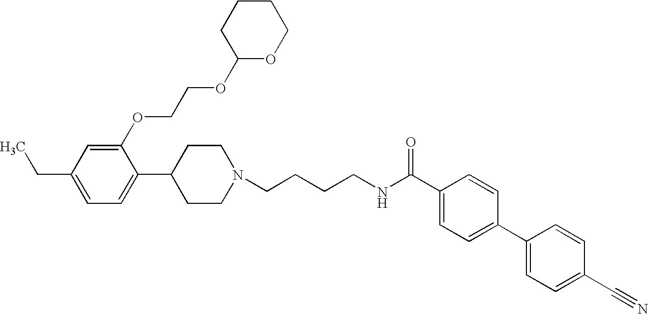

The same method was employed as in the preparation of intermediate 9 but starting from the available 4′-cyano-biphenyl-4-carboxylic acid and gave the title compound as a yellow oil in a 20% yield; LC/MS: M+H C₃₈H₄₈N₃O₄ 610.

### Intermediate 11: 1-(4-{1-[2-(Tetrahydro-pyran-2-yloxy)-ethoxy]-5,6,7,8-tetrahydro-naphthalen-2-yl}-piperidin-1-yl)ethanone

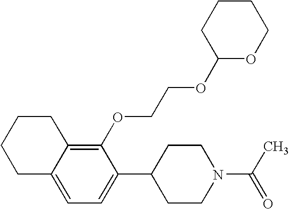

The same method was employed as in the preparation of intermediate 5 but starting from intermediate 2 and gave the title compound as a yellow oil in a 95% yield; LC/MS: M+H C₂₄H₃₆NO₄ 402.

### Intermediate 12: 4-{1-[2-(Tetrahydro-pyran-2-yloxy)-ethoxy]-5,6,7,8-tetrahydro-naphthalen-2-yl}-piperidine

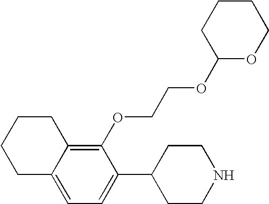

The same method was employed as in the preparation of intermediate 6 but starting from intermediate 11 and gave the title compound as a yellow oil in a quantitative yield; LC/MS: M+H C₂₂H₃₄NO₃ 360.

### Intermediate 13: 2-[4-(4-(4-{1-[2-(Tetrahydro-pyran-2-yloxy)-ethoxy]-5,6,7,8-tetrahydro-naphthalen-2-yl}-piperidin-1-yl)-butyl}-isoindole-1,3-dione

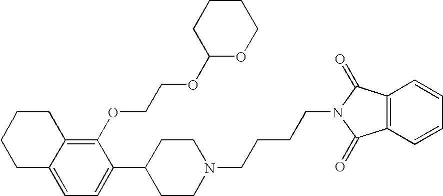

The same method was employed as in the preparation of intermediate 7 but starting from intermediate 12 and gave the title compound as a yellow oil in a 93% yield; LC/MS: M+H C₃₄H₄₅N₂O₅ 561.

### Intermediate 14: 4-(4-{1-[2-(Tetrahydro-pyran-2-yloxy)-ethoxy]-5,6,7,8-tetrahydro-naphthalen-2-yl}-piperidin-1-yl)-butylamine

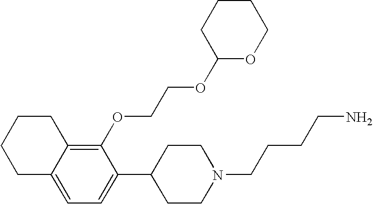

The same method was employed as in the preparation of intermediate 8 but starting from intermediate 13 and gave the title compound as a yellow oil (contained 30% of formed pyridazine dione); LC/MS: M+H C₂₆H₄₃N₂O₃431.

### Intermediate 15: 4-Trifluoromethyl-thiobenzamide

A solution of α,α,α-trifluoro-p-tolunitrile (603.5 g, 3.53 mol) in dry DMF (2 L) under N₂ was heated at 70° C. and the thioacetamide (505 g, 1.9 eq.) was added. The reaction mixture was treated with HCl gas for 15 minutes and stirred at 95° C. for 6 hours. This treatment was repeated 3 times and the mixture stirred at rt for 24 hours. After cooling at 0° C., water was added and the residue extracted with diethyl ether (4 L). The organic layer was washed with water (3 L), dried over Na₂SO₄ and the solvent evaporated. The brownish powder was washed with pentane (3 L) to give the title compound (530.3 g, 2.59 mol) as a brown solid in a 73% yield; GC/MS: M⁺ C₈H₆F₃NS 205.

### Intermediate 16: 4-Methyl-2-(4-trifluoramethyl-phenyl)-thiazole-5-carboxylic acid ethyl ester

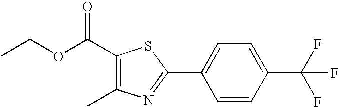

To a solution of intermediate 15 (530.3 g, 2.59 mol) in EtOH (2.6 L) was added 2-chloro-3-oxo-butyric acid ethyl ester (465 mL, 1.3 eq.) and the mixture was stirred at rt for 7 hours and at 70° C. for 14 hours. After cooling at 0° C., the precipitate was filtered and washed with cold EtOH (500 mL) to give the title compound (573.0 g, 1.89 mol) as a beige powder in a 73% yield; ¹H NMR (CDCl₃, 300 MHz) [symbol] 7.9 (d, 2H), 7.6 (d, 2H), 4.3 (q, 2H), 2.65 (s, 3H), 1.25 (t, 3H).

### Intermediate 17: 4-Bromomethyl-2-(4-trifluoromethyl-phenyl)-thiazole-5-carboxylic acid ethyl ester

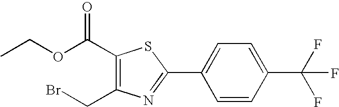

To a solution of intermediate 16 (15.75 g, 50.0 mmol) in CCl₄ was added slowly N-bromosuccinimide (8.9 g, 1.1 eq.) and AlBN (1 g, 10% mol) and the mixture was stirred at 80° C. for 3 hours. The mixture was filtered and the filtrate evaporated. After purification by flash chromatography, using DCM/Cyclohexane (60/40) as eluent, the title compound (4.9 g, 12.5 mmol) was obtained as white solid in a 25% yield. ¹H NMR (CDCl₃, 300 MHz) [symbol] 8.2 (d, 2H), 7.8 (d, 2H), 5.1 (s, 2H), 4.5 (q, 2H), 1.3 (t, 3H)

### Intermediate 18: 4-Acetoxymethyl-2-(4-trifluoromethyl-phenyl)-thiazole-5-carboxylic acid ethyl ester

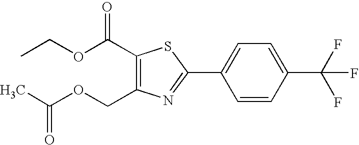

To a solution of intermediate 17 (4.9 g, 12.5 mmol) in AcOH (15 mL) was added sodium acetate (2.0 g, 2 eq.) and the mixture was stirred at reflux for 14 hours. After cooling to rt, the mixture was diluted with water (150 mL) and extracted with diethyl ether (250 mL). The organic layer was washed with a 1 N NaOH solution, dried over Na₂SO₄ and the solvent evaporated. The title compound (3.24 g, 8.7 mmol) was obtained as white crystals in a 72% yield; m.p. 82° C.

### Intermediate 19: 4-Hydroxymethyl-2-(4-trifluoromethyl-phenyl)-thiazole-5-carboxylic acid

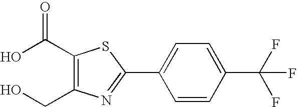

To a solution of intermediate 18 (3.24 g, 8.7 mmol) in EtOH/H₂O (40 mL/20 mL) was added NaOH (1.4 g, 4 eq.) and the mixture was stirred at reflux for 2 hours. After partial evaporation, water (100 mL) was added and treated with a concentrated HCl solution to obtain pH=1. The precipitate was filtered off, washed with water and dried to give the title compound (2.38 g, 7.8 mmol) as white solid in a 90% yield; m.p. 250-252° C.

### Intermediate 20: 4-Hydroxymethyl-2-(4-trifluoromethyl-phenyl)-thiazole-5-carboxylic acid [4-(4-{1-2-(tetrahydro-pyran-2-yloxy)-ethoxy]-5,6,7,8-tetrahydro-naphthalen-2-yl}-piperidin-1-yl)-butyl]-amide

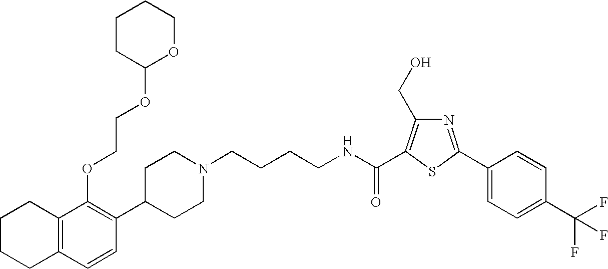

The same method was employed as in the preparation of intermediate 9 but starting from intermediate 14 and intermediate 19 and gave the title compound as a beige powder in a 28% yield; m.p. 146-148° C.

### Intermediate 21: 4′-Cyano-biphenyl-4-carboxylic acid [4-(4-{1-[2-(tetrahydro-pyran-2-yloxy)-ethoxy)-5,6,7,8-tetrahydro-naphthalen-2-yl}-piperidin-1-yl)-butyl]-amide

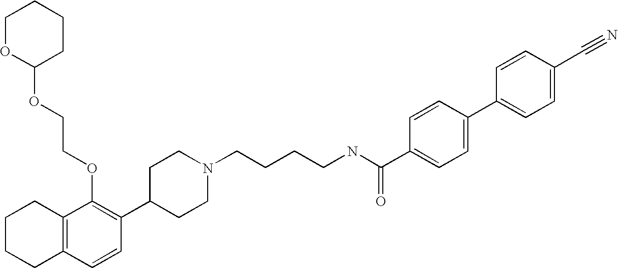

The same method was employed as in the preparation of intermediate 9 but starting from intermediate 14 and 4′-cyano-biphenyl-4-carboxylic acid and gave the title compound as white oil in a 62% yield; LC/MS: M+H C₄₀H₄₉N₃O₄ 636.

### Intermediate 22: 1-[4-(1-Benzyloxy-5,6,7,8-tetrahydro-naphthalen-2-yl)-piperidin-1-yl]-ethanone

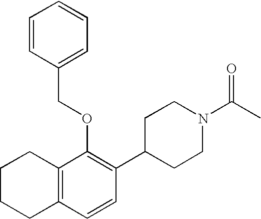

A solution of intermediate 2 (11 g, 40 mmol), K₂CO₃ (19.0 g, 1.5 eq.) in methyl ethyl ketone (150 mL) was stirred at 80° C. for 10 minutes. Benzyl bromide (7.7 g, 1.1 eq.) was added and the mixture was stirred to reflux for 2.5 hours. After filtration, the filtrate was evaporated and the residue washed with water, extracted with ether and evaporated off. The solid was washed with iPr₂O to give the title compound (9.5 g, 26.2 mmol) as beige crystals in a 66% yield; m.p. 108-110° C.

### Intermediate 23: 4-(1-Benzyloxy-5,6,7,8-tetrahydro-naphthalen-2-yl)-piperidine

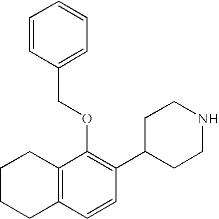

The same method was employed as in the preparation of intermediate 6 but starting from intermediate 22 and gave the title compound which was used directly in the next step without purification; LC/MS: M+H C₂₂H₂₈NO 322.

### Intermediate 24: 2-{4-[4-(1-Benzyloxy-5,6,7,8-tetrahydro-naphthalen-2-yl)-piperidin-1-yl]-butyl}-isoindole-1,3-dione

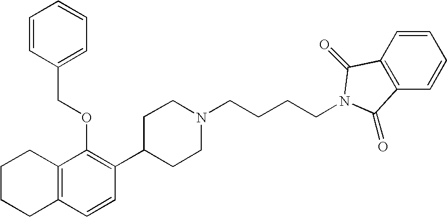

The same method was employed as in the preparation of intermediate 7 but starting from intermediate 23 and gave the title compound as an oil in a quantitative yield directly used in the next step without purification; LC/MS: M+H C₃₄H₃₉N₂O₃ 523.

### Intermediate 25: 4-[4-(1-Benzyloxy-5,6,7,8-tetrahydro-naphthalen-2-yl)-piperidin-1-yl]-butylamine

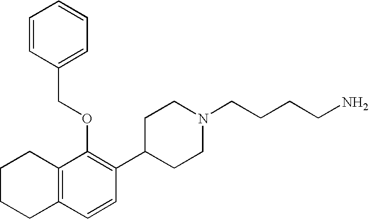

The same method was employed as in the preparation of intermediate 8 but starting from intermediate 24 and gave the title compound as an oil which was directly used in the next step without purification; LC/MS: M+H C₂₆H₃₇N₂O 393.

### Intermediate 26: 4′-Trifluoromethyl-biphenyl-4-carboxylic acid {4-[4-(1-benzyloxy-5,6,7,8-tetrahydro-naphthalen-2-yl)-piperidin-1-yl]-butyl}-amide

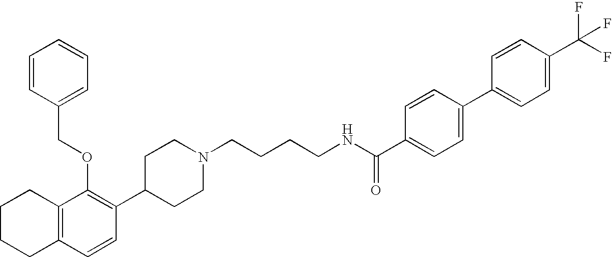

The same method was employed as in the preparation of intermediate 9 but starting from intermediate 25 and 4′-trifluoromethyl-biphenyl-4-carboxylic acid and gave the title compound as white crystals in 45% yield after recrystallisation in CH₃CN; m.p. 169-170° C.

### Intermediate 27: 4′-Trifluoromethyl-biphenyl-4-carboxylic acid {4-[4-(1-hydroxy-5,6,7,8-tetrahydro-naphthalen-2-yl)-piperidin-1-yl]-butyl}-amide

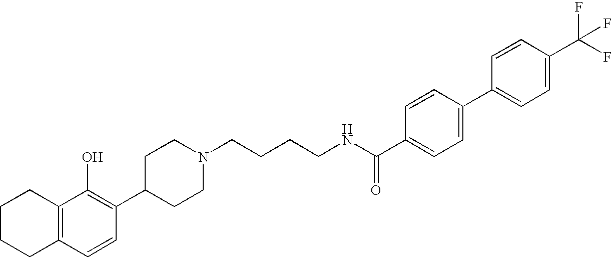

To a mixture of intermediate 26 (4.0 g, 6.2 mmol) in EtOH (150 mL) was added Pd/C, 10% (0.6 g) and the reaction mixture was stirred under an atmospheric pressure of hydrogen at 60° C. for 2 hours. The mixture was filtered through a bed of celite. The filtrate was evaporated under reduced pressure to give the title compound (3 g, 5.4 mmol) as a white crystals after crystallisation from EtOH; m.p. 220-222° C.

### Intermediate 28: 2-(4-Chloro-phenyl)-4-methyl-thiazole-5-carboxylic acid [4-(4-{1-[2-(tetrahydro-pyran-2-yloxy)-ethoxy]-5,6,7,8-tetrahydro-naphthalen-2-yl}piperidin-1-yl)-butyl]-amide

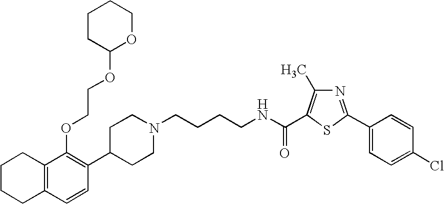

To a solution of intermediate 14 (1.0 g, 2.33 mmol) in DMF (50 mL) was added the available 2-(4-chloro-phenyl)-4-methyl-thiazole-5-carboxylic acid (0.530 g, 0.9 eq.), HOBT (0.38 g, 1.2 eq.), EDCl (0.535 g, 1.2 eq.) and TEA (390 μL, 1.2 eq.) and the reaction mixture was stirred at rt for 48 hours. After evaporation of the DMF, the product was dissolved in DCM. The organic layer was washed with a saturated NaHCO₃ solution, dried over Na₂SO₄, filtered and evaporated off. Purification by flash chromatography using DCM/MeOH 95/5 as eluent gave the title compound as a brown oil (0.815 g, 1.23 mmol) in a 53% yield; LC/MS: M+H C₃₇H₄₉ClN₃O₄S 666.

### Intermediate 29: 2-Bromo-4-methyl-thiazole-5-carboxylic acid ethyl ester

A solution of 2-methyl-1-nitrosooxy-propane (28.2 mL, 2.1 eq.) in CH₃CN (700 mL) was cooled to 0° C. and bromo-trimethyl-silane (32 mL, 2.1 eq.) was added dropwise over 20 min. A solution of the available 2-amino-4-methyl-thiazole-5-carboxylic acid ethyl ester (18.6 g, 0.1 mol) in a mixture CH₃CN/EtOAc: 75/25 heated to 55° C. was added dropwise over 45 min. During the addition the reaction was maintained at 0° C. and then allowed to warm to rt and stirred for 2 hours. After evaporation, the product was extracted with AcOEt, washed with water, dried over Na₂SO₄, filtered and evaporated. The title compound was obtained as a yellow solid (21.74 g, 86.96 mmol) in a 87% yield; GC/MS: M⁺ C₇H₈BrNO₂S 250.

### Intermediate 30: 2-(4-Cyano-phenyl)-4-methyl-thiazole-5-carboxylic acid ethyl ester

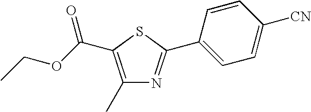

To a solution of intermediate 29 (10.0 g, 40 mmol) in DMF (200 mL) was added Pd(PPh₃)₄ (2.76 g, 0.06 eq.), a 2M Na₂CO₃ solution (50 mL, 2.5 eq.) and 4-cyanophenyboronic acid (11.68 g, 2.0 eq.) and the mixture was stirred at 100° C. for 2 days. After evaporation of DMF, water was added and the product was extracted with DCM and the organic layer was filtered through a bed of celite. The filtrate was dried over Na₂SO₄ and evaporated. The title compound was obtained as a white solid (10.1 g, 37 mmol) in a 92.8% yield after purification by flash chromatography using DCM/cyclohexane 80/20 and 90/10 as eluent; GC/MS: M+ C₁₄H₁₂N₂O₂S 272.

### Intermediate 31: 2-(4-Cyano-phenyl)₄-methyl-thiazole-5-carboxylic acid

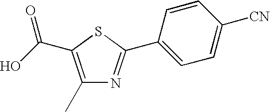

A suspension of intermediate 30 (4.0 g, 14.7 mmol) in EtOH (150 mL) was treated with LiOH.H₂O (1.23 g, 2.0 eq.) and the mixture was stirred at rt for 2 days. After evaporation, the residue was acidified with a 1N HCl solution. The resulting precipitate was filtered and dried. The title compound was obtained as a white solid (4.0 g, 16.39 mmol) in a quantitative yield; m.p. 265-270° C.

### Intermediate 32: 2-(4-Cyano-phenyl)-4-methyl-thiazole-5-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

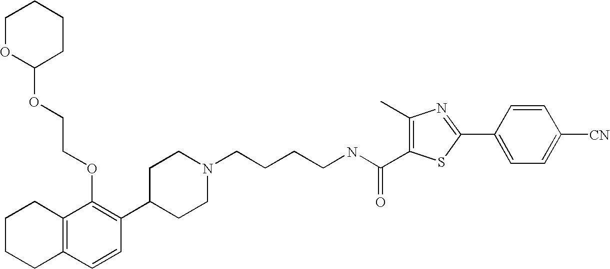

The same method was employed as in the preparation of intermediate 28 but starting from intermediate 14 and intermediate 31 and gave the title compound as a brown foam in a 39.5% yield after purification by flash chromatography using DCM/MeOH 90/10 as eluent; LC/MS: M+H C₃₈H₄₉N₄O₄S 657.

### Intermediate 33: 2-(2,4-Dichloro-phenyl)-4-methyl-thiazole-5-carboxylic acid ethyl ester

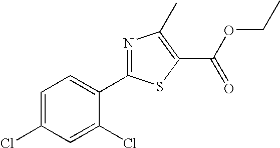

A solution of 2,4-dichloro-benzamide (19.0 g, 0.1 mol), P₄S₁₀ (8.88 g, 0.2 eq.) and NaHCO₃ (16.8 g, 2.0 eq.) in toluene (200 mL) was heated under reflux for 1 hour. After concentration, the residue was purified through a bed of silica using DCM/AcOEt 80/20 as eluent. The isolated 2,4-dichloro-thiobenzamide (5.0 g, 24.26 mmol) was dissolved in EtOH and 2-chloro-3-oxo-butyric acid ethyl ester (3.3 mL, 1 eq.) in EtOH was added. The reaction was heated under reflux for 2 days. On cooling to rt, the reaction mixture was filtered to give the title compound as pink crystals (1.57 g, 4.96 mmol) in a 5% global yield; m.p. 100° C.

### Intermediate 34: 2-(2,4-Dichloro-phenyl)-4-methyl-thiazole-5-carboxylic acid

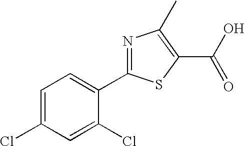

A suspension of intermediate 33 (1.56 g, 4.95 mmol) in EtOH (100 mL) was treated with a 1N NaOH solution (15 mL, 3.0 eq.) and the reaction mixture was refluxed for 2 hours. After evaporation, the residue was acidified with a 1N HCl solution. The resulting precipitate was filtered washed with water and dried. The title compound was obtained as a white solid (1.42 g, 4.95 mmol) in a quantitative yield; LC/MS: M+H C₁₁H₈Cl₂NO₂S 288.

### Intermediate 35: 2-(2,4-Dichloro-phenyl)-4-methyl-thiazole-5-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

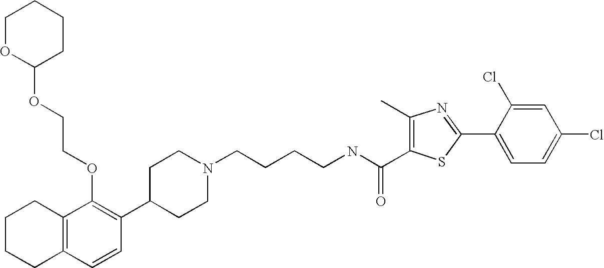

The same method was employed as in the preparation of intermediate 28 but starting from intermediate 14 and intermediate 34 and gave the title compound as a yellow oil in a 41% yield after purification by flash chromatography using DCM/MeOH 95/5 as eluent; LC/MS: M+H C₃₇H₄₈Cl₂N₃O₄S 700.

### Intermediate 36: 2′,4′-dichloro-biphenyl-4-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

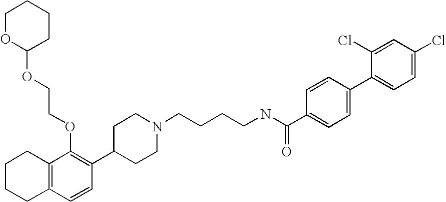

The same method was employed as in the preparation of intermediate 28 but starting from intermediate 14 and the available 2′,4′-dichloro-biphenyl-4-carboxylic acid and gave the title compound as beige crystals in a 43% yield after purification by flash chromatography using DCM/MeOH 95/5 as eluent; ¹H NMR (CDCl3, 300 MHz) [symbol] 8.56 (t, 1H), 7.93 (d, 2H), 7.7 (m, 1H), 7.5 (m, 4H), 6.96 (d, 1H), 6.8 (d, 1H), 4.7 (m, 1H), 3.89-1.49 (m, 37H).

### Intermediate 37: 4′-Chloro-biphenyl-4-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

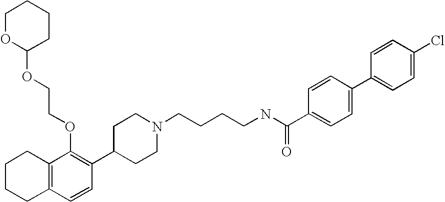

The same method was employed as in the preparation of intermediate 28 but starting from intermediate 14 and the available 4-chloro-biphenyl-4-carboxylic acid and gave the title compound as beige crystals in a 36% yield after purification by flash chromatography using DCM/MeOH 95/5 as eluent; LC/MS: M+H C₃₉H₅₀ClN₂O₄ 645.

### Intermediate 38: 2-(4-Cyano-phenyl)-4-methyl-oxazole-5-carboxylic acid

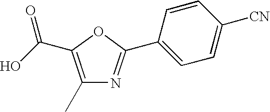

A solution of the available 2-(4-cyano-phenyl)-4-methyl-oxazole-5-carboxylic acid methyl ester (1.0 g, 4.13 mmol) in THF (50 mL) was treated with a 1N NaOH solution (4 mL, 0.95 eq.) and the reaction was stirred at rt for 3 days. The reaction was neutralised with 1N HCl solution and the solvent was evaporated. The residue was washed with water. The precipitate was filtered, washed with water and dried to give the title compound as a white solid (0.94 g, 4.13 mmol) in a quantitative yield; LC/MS: M+H C₁₂H₉N₂O₃ 229.

### Intermediate 39: 2-(4-Cyano-phenyl)-4-methyl-oxazole-5-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

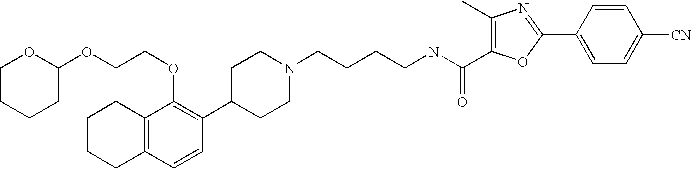

The same method was employed as in the preparation of intermediate 28 but starting from intermediate 14 and intermediate 38 and gave the title compound as a dark yellow oil in a 40% yield after purification by flash chromatography using DCM/MeOH 93/7 and 85/15 as eluents; LC/MS: M+H C₃₈H₄₉N₄O₅ 641.

### Intermediate 40: N-(4-Bromo-phenyl)-methanesulfonamide

To a solution of 4-bromo-phenylamine (8.60 g, 50 mmol) and TEA (10.35 g, 2.05 eq.) in DCM (100 mL) cooled to −78° C. was slowly added a solution of methanesulfonyl chloride (6.01 g, 1.05 eq.) in DCM. The mixture was warmed to rt and the mixture stirred overnight. Water was added and the mixture was decantated. The aqueous layer was extracted with DCM and the organic layer was dried over Na₂SO₄, filtered and evaporated off. The title compound was obtained as a white solid (6.75 g, 27 mmol) in a 54% yield after purification by flash chromatography using DCM as eluent; m.p. 140-142° C.

### Intermediate 41: 4′-Methanesulfonylamino-biphenyl-4-carboxylic acid methyl ester

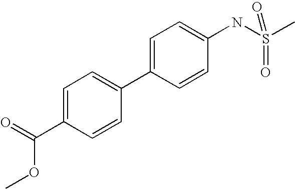

The same method was employed as in the preparation of intermediate 30 but starting from intermediate 40 and the available 4-methoxycarbonylphenylboronic acid and gave the title compound as a pale grey solid in a 33.2% yield after recrystallisation from CH₃CN; m.p. 201-203° C.

### Intermediate 42: 4-Methanesulfonylamino-biphenyl-4-carboxylic acid

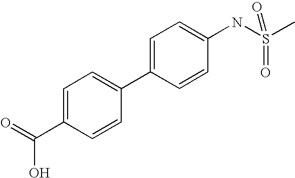

The same method was employed as in the preparation of intermediate 34 but starting from intermediate 41 and gave the title compound as a white solid in a quantitative yield. This intermediate was directly used in the next step; LC/MS: M−H C₁₄H₁₂NO₄S 290

### Intermediate 43: 4′-Methanesulfonylamino-biphenyl-4-carboxylic acid [4-(4-{1-[2-(tetrahydro-pyran-2-yloxy)-ethoxy]-5,6,7,8-tetrahydro-naphthalen-2-yl}-piperidin-1-yl)-butyl]-amide

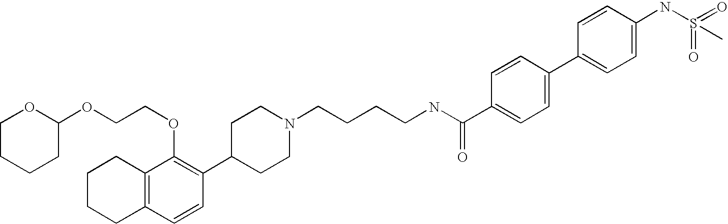

The same method was employed as in the preparation of intermediate 28 but starting from intermediate 14 and intermediate 42 and gave the title compound as a yellow oil in a 26% yield after purification by flash chromatography using DCM/MeOH 85/15 as eluent. LC/MS: M+H C₄₀H₅₄N₃O₆S 704.

### EXAMPLES

### Example 1

### 4′-Trifluoromethyl-biphenyl-4-carboxylic acid (5-{4-[4-ethyl-2-(2-hydroxy-ethoxy)-phenyl]-piperidin-1-yl}-butyl)-amide hydrochloride

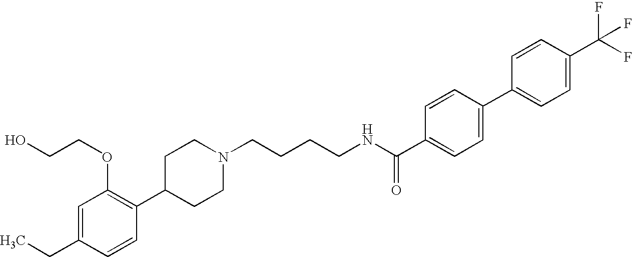

To a solution of intermediate 9 (0.28 g, 0.43 mmol) in MeOH/DCM 50/50 (20 mL) was added 1N HCl (0.9 ml, 2 eq.) and the resulting mixture stirred at rt for 24 hours. The solvent was evaporated and the residue treated with DCM/iPr₂O to give the title compound (0.080 g, 0.13 mmol) as white crystals in 31% yield; ¹H NMR (CDCl₃, 300 MHz) [symbol] 8.25 (d, 2H), 7.8 (d, 6H), 7.15 (d, 1H), 6.8 (m, 2H), 4.2 (m, 4H), 3.8 (m, 4H), 3.2 (m, 2H), 3-1.8 (m, 15H), 1.3 (t, 3H); LC/Tof: ES⁺ 569.2946 7.8 ppm.

### Example 2

### 4′-Cyano-biphenyl-4-carboxylic acid (5-{4-[4-ethyl-2-(2-hydroxy-ethoxy)-phenyl]-piperidin-1-yl}-butyl)-amide hydrochloride

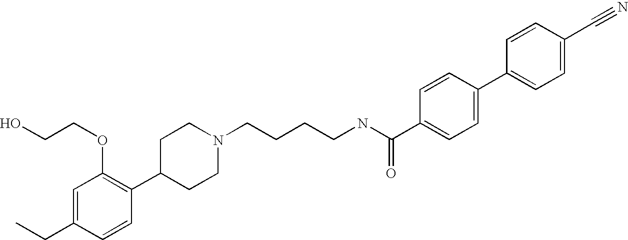

The same method was employed as in the preparation of example 1 but starting from intermediate 10 and gave the title compound as white crystals in a 8% yield after recrystallisation in iPr₂O; LC/Tof: ES⁺ 526.3093 4.6 ppm; ¹H NMR (CDCl₃, 300 MHz) [symbol] 8.1 (d, 2H), 7.7 (d, 6H), 6.95 (d, 1H), 6.7 (m, 2H), 4.2 (m, 4H), 4.0 (m, 4H), 3.4 (m, 2H), 3.1-1.6 (m, 15H), 1.2 (t, 3H).

### Example 3

### 4-Hydroxymethyl-2-(4-trifluoromethyl-phenyl)-thiazole-5-carboxylic acid (4-{4-[1-(2-hydroxy-ethoxy)-5,6,7,8-tetrahydro-naphthalen-2-yl]-piperidin-1-yl}-butyl)-amide

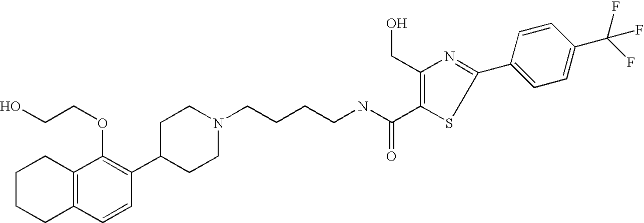

The same method was employed as in the preparation of example 1 but starting from intermediate 20 and gave the title compound as beige crystals in a 39% yield after recrystallisation in CH₃CN; m.p. 157-159° C.; LC/MS: M+H C₃₃H₄₁F₃N₃O₄S 632.

### Example 4

### 4′-Trifluoromethyl-biphenyl-4-carboxylic acid (4-{4-[1-(2-ethoxy-ethoxy)-5,6,7,8-tetrahydro-naphthalen-2-yl]-piperidin-1-yl}-butyl)-amide

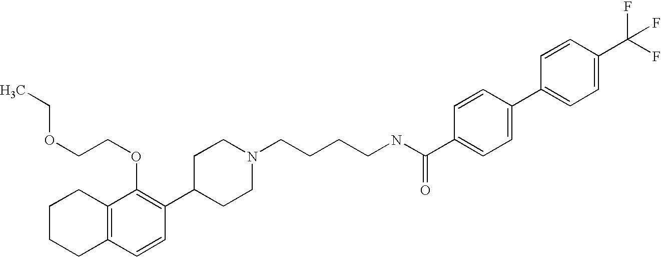

To a solution of TMAD (2.0 eq.) in dry THF under argon was added tributylphosphine (2.2 eq.) and the mixture was stirred at rt for 10 minutes. Intermediate 27 (1.0 eq) and 2-ethoxy-ethanol (1.2 eq.) were added and the mixture was stirred at rt for 48 hours. Water was added and the reaction mixture evaporated. The residue was taken up into water and the mixture filtered. After recrystallisation in CH₃CN, the title compound was obtained as white crystals in a 81% yield; m.p. 190° C.; LC/Tof: ES⁺ 623.3485 3.8 ppm.

### Example 5

### 4′-Cyano-biphenyl-4-carboxylic acid (4-{4-[1-(2-hydroxy-ethoxy)-5,6,7,8-tetrahydro-naphthalen-2-yl]-piperidin-1-yl}-butyl)-amide hydrochloride

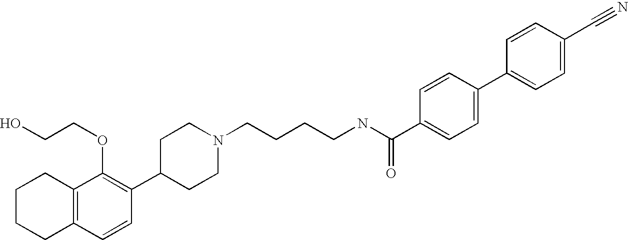

The same method was employed as in the preparation of example 1 but starting from intermediate 21 and gave the title compound as white crystals in a 77% yield after recrystallisation in iPrO₂; m.p. 174° C.; LC/tof: ES+552.3176 9 ppm.

### Example 6

### 2-(4-Chloro-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide

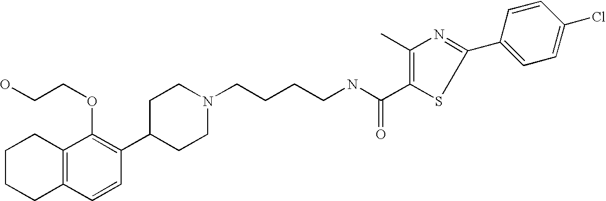

A solution of intermediate 28 (0.815 g, 1.23 mmol) in MeOH (80 mL) was treated with toluene-4-sulfonic acid (small quantity) and stirred at reflux for 70 hours. After evaporation, the product was dissolved with DCM and washed with a 1N NaOH solution. The organic layer was dried over Na₂SO₄, filtered and evaporated. The title compound was obtained as a white solid (0.62 g, 1.06 mmol) in a 87% yield; m.p. 198° C.; LC/Tof: ES+ Calculated, 582.2557; Found, 582.2567 1.8 ppm.

### Example 7

### 2-(4-Cyano-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide, hydrochloride

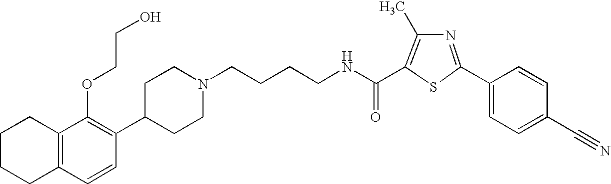

A solution of intermediate 32 (0.6 g, 0.92 mmol) in DCM/MeOH (10 mL/10 mL) was treated with 1N HCl and the reaction mixture was stirred at rt for 24 hours. After concentration to dryness, acetone was added. The resulting precipitate (hydrochloride form) was filtered and triturated with IprO₂. The title compound was obtained as a beige solid (0.51 g, 0.84 mmol) in a 91.6% yield; m.p. 188° C.; LC/MS: M+H C₃₃H₄₁N₄O₃S 573.

### Example 8

### 2-(2,4-dichloro-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide

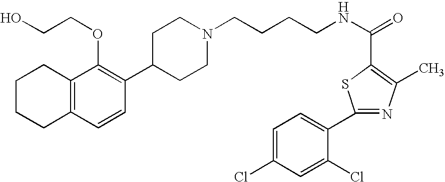

The same method was employed as in the preparation of example 6 but starting from intermediate 35 and gave the title compound as a white solid in a 44% yield after recristallisation from IprO₂; m.p. 122-124° C.; LC/Tof: ES+ calculated, 616.2167; found, 616.2160-1.1 ppm.

### Example 9

### 2′,4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide

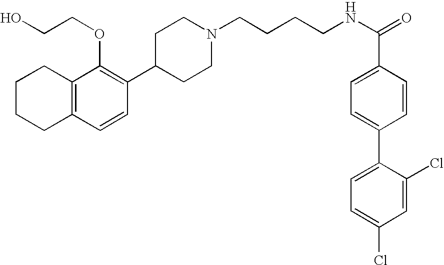

The same method was employed as in the preparation of example 6 but starting from intermediate 36 and gave the title compound as a white solid in a 72% yield after recristallisation from CH₃CN; m.p. 145° C.; LC/Tof: ES+ Calculated 595.2494; Found, 595.2516 3.7 ppm.

### Example 10

### 4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide

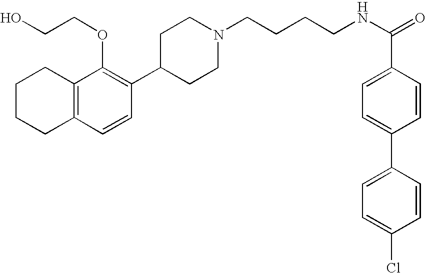

The same method was employed as in the preparation of example 6 but starting from intermediate 37 and gave the title compound as a white solid in a 73% yield after recrystallisation from CH₃CN; m.p. 162° C.; LC/Tof: ES+ Calculated 561.2884; Found 561.2886 0.3 ppm.

### Example 11

### 2-(4-Cyanophenyl)-4-methyl-oxazole-5-carboxylic acid (4-{4-[1-(2-hydroxy-ethoxy)-5,6,7,8-tetrahydro-naphthalen-2-yl]-piperidin-1-yl}-butyl)-amide

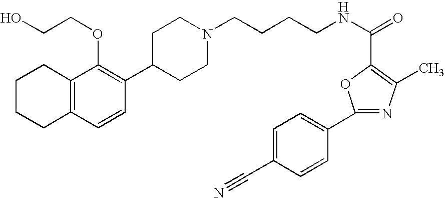

The same method was employed as in the preparation of example 6 but starting from intermediate 39 and gave the title compound as a yellow solid in a 50% yield after cristallisation from IprO₂; m.p. 109-111° C.; LC/Tof: ES+ Calculated, 557.3127; Found, 557.3069-10.4 ppm.

### Example 12

### 4′-Methanesulfonylamino-biphenyl-4-carboxylic acid (4-{4-[1-(2-hydroxy-ethoxy)-5,6,7,8-tetrahydro-naphthalen-2-yl]-piperidin-1-yl}-butyl)-amide

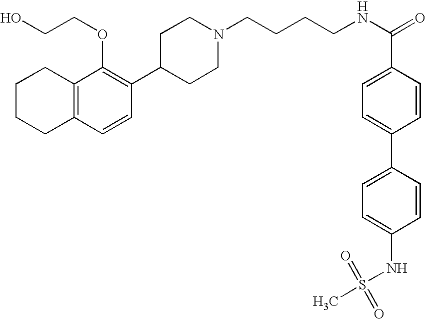

The same method was employed as in the preparation of example 6 but starting from intermediate 43 and gave the title compound as a yellow solid in a 41% yield purification by flash chromatography using DCM/MeOH 85/15 as eluents; m.p. 216° C.; LC/MS: M+H C₃₅H₄₆N₃O₅S 620.

Biological Assays

The Examples were tested in vivo and/or in vitro according to the following assay methods.

In Vitro Assay

HepG₂ cells, stably transfected with a construct comprising the LDL-r promoter and the luciferase reporter gene, were seeded at 50.000 cells/well in 96 well plates. After 1 day, cells were incubated with compounds for 24 hours in RPMI medium containing 2% of lipoprotein-deficient serum. Compounds were tested from 10⁻⁸M to 10⁻⁹M. Cell lysates were prepared and the luciferase activity was measured by the luciferase assay system (Promega). Induction of luciferase activity was calculated taking untreated cells as control. The ED₅₀ of each compounds was determined compared to the ED₅₀ of an internal standard.

In Vivo Assay

Compounds were prepared for oral administration by milling with 0.5% hydroxypropylmethylcellulose and 5% Tween 80. Hamsters were fed for 2 weeks with a diet containing 0.2% of cholesterol and 10% of coconut oil. Then compounds were administered once a day for 3 days, from 20 to 0.2 mg/kg. Plasma lipid levels including total cholesterol, VLD/LDL cholesterol, VLD/LDL triglycerides and HDL-cholesterol were determined after ultracentrifugation (density 1.063 g/ml to separate VLD/LDL fraction and HDL fraction) using the Biomerieux enzymatic kit. Reductions in VLD/LDL cholesterol and TG plasmatic levels were calculated taking solvent treated animals as control and ED₅₀ of each compound was determined.

The compounds of the invention are potent and specific inducers of LDL-r expression.

Using the above in vitro assay all Examples of the invention induced luciferase activity having EC₅₀ values in the range 1 nM to 64 nM.

2-(2,4-dichloro-phenyl)-4-methyl-thiazole-5-carboxylic acid (4-{4-[1-(2-hydroxy-ethoxy)-5,6,7,8-tetrahydro-naphthalen-2-yl]-piperidin-1-yl}butyl)-amide (Example 8) had an EC₅₀ value of 13 nM.

4′-Methanesulfonylamino-biphenyl-4-carboxylic acid (4-{4-[1-(2-hydroxy-ethoxy)-5,6,7,8-tetrahydro-naphthalen-2-yl]-piperidin-1-yl}-butyl)-amide (Example 12) had an EC₅₀ value of 5 nM.

1. A compound of formula (I) 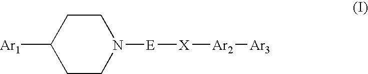
wherein
Ar₁ is:
(i) phenyl, naphthyl or phenyl fused by a C₃₋₈cycloalkyl; or
(ii) heterocyclyl selected from the group consisting of:
monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated or aromatic, provided that at least one ring is aromatic;
where Ar₁ is independently substituted by at least one R¹ group and is independently substituted by 0-3 R³ groups;
Ar₂ is a phenyl group, a 5-6 membered heteroaromatic group or a bicyclic heteroaromatic group, each of which are optionally substituted by 1-4 groups independently selected from the list: C₁₋₄alkyl, halogen, hydroxy, C₁₋₄alkoxy, C₁₋₆acyl, C₁₋₆acyloxy, amino, C₁₋₄alkylamino, di-C₁₋₄alkylamino, —(CH₂)ₙOH, —(CH₂)ₙNRₓR_{y}, O(CH₂)ₙO(CH₂)ₘOR², —O(CH₂)ₙC(O)NRₓR_{y}, —O(CH₂)ₙCN, C₂-alkenyl, —O(CH₂)ₙCO₂R²—OSO₂(CH₂)ₚCH₃, —OSO₂NRₓR_{y} and —CO₂(CH₂)ₚCH₃;
Ar₃ is:
(i) phenyl, naphthyl or phenyl fused by a C₃₋₈cycloalkyl; or
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic;
wherein Ar₃ is optionally substituted by 1-4 groups independently selected from the group consisting of: hydroxy, C₁₋₄alkyl, C₁₋₄alkoxy, C₂₋₄alkenyl, C₂₋₄alkenyloxy, C₁₋₄perfluoroalkoxy, C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, nitro, C₁₋₄perfluoroalkyl, C₁₋₄alkylcarbonyl, fluoroC₁₋₄alkylcarbonyl, C₁₋₄alkoxycarbonyl, aminocarbonyl, C₁₋₄alkylaminocarbonyl, di-C₁₋₄alkylaminocarbonyl, C₁₋₄alkylsulfonyl, C₁₋₄alkylaminosulfonyl, di-C₁₋₄alkylaminosulfonyl, C₁₋₄alkylsulfonyl and C₁₋₄alkylsulfoxy;
E is —C₁₋₁₆alkylene-;
X is —CONR²— or —NR²CO—;
wherein
R¹ is O(CH₂)ₙOR²;
R² is C₁₋₄alkyl or hydrogen;
R³ is halogen, C₁₋₄alkoxy or C₁₋₄alkyl;
Rₓ and R_{y} are independently C₁₋₄alkyl or hydrogen;
n and m are independently 1-4; and
p is 0-4;
or a physiologically acceptable prodrug, salt or solvate thereof.

2. A compound according to claim 1 wherein Ar₁ is phenyl, naphthyl, 1,2,3,4-tetrahydronaphthyl, indolyl, benzofuranyl, benzothiophenyl or indazolyl.

3. A compound according to claim 1 wherein E is n-butylene.

4. A compound according to claim 1 wherein X is —NR²CO—.

5. A compound according to claim 1 wherein Ar₂ is phenyl or a 5-6-membered heteroaromatic group.

6. A compound according to claim 5 wherein Ar₂ is optionally substituted by C₁₋₄alkyl, halogen, hydroxy, hydroxyC₁₋₄alkyl or C₁₋₄alkoxy.

7. A compound according to claim 1 wherein Ar₃ is phenyl, pyridyl or thienyl.

8. A compound according to claim 7 wherein Ar₃ is substituted by halogen, C₁₋₄perfluoroalkyl, nitrile, C₁₋₄acyl, C₁₋₄alkylsulfonyl, or C₁₋₄alkylsulfonylamino.

9. A compound according to claim 1 wherein R¹ is —OCH₂CH₂OR².

10. A compound according to claim 1 wherein
Ar₁ is phenyl, naphthyl, 1,2,3,4-tetrahydronaphthyl, indolyl, benzofuranyl, benzothiophenyl or indazolyl; where Ar₁ is independently substituted by at least one R¹ group and is independently substituted by 0-3 R³ groups;
Ar₂ is phenyl, pyridyl, thiazolyl, oxazolyl or imidazolyl, each of which are optionally substituted by 1-4 groups independently selected from the group; C₁₋₄alkyl, halogen, hydroxy, hydroxyC₁₋₄alkyl and C₁₋₄alkoxy;
Ar₃ is phenyl, pyridyl or thienyl, wherein Ar₃ is optionally substituted by 1-4 groups independently selected from the group consisting of: halogen, C₁₋₄perfluoroalkyl, nitrile, C₁₋₄acyl, C₁₋₄alkylsulfonyl, and C₁₋₄alkylsulfonylamino;
E is n-butylene;
X is —NR²CO—;
wherein
R¹ is —OCH₂CH₂OR²;
R² is C₁₋₄alkyl or hydrogen;
R³ is halogen, C₁₋₄alkoxy or C₁₋₄alkyl; and
n is 1-4.

11. A compound according to claim 1 selected from the group consisting of:
4-Hydroxymethyl-2-(4-trifluoromethyl-phenyl)-thiazole-5-carboxylic acid (4-{4-[1-(2-hydroxy-ethoxy)-5,6,7,8-tetrahydro-naphthalen-2-yl]-piperidin-1-yl}-butyl)-amide;
4′-Cyano-biphenyl-4-carboxylic acid (4-{4-[1-(2-hydroxy-ethoxy)-5,6,7,8-tetrahydro-naphthalen-2-yl]-piperidin-1-yl}-butyl)-amide hydrochloride;
2-(4-Cyano-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide, hydrochloride;
2-(2,4-dichloro-phenyl)-4-methyl-thiazole-5-carboxylic acid (4-{4-[1-(2-hydroxy-ethoxy)-5,6,7,8-tetrahydro-naphthalen-2-yl]-piperidin-1-yl}-butyl)-amide;
2′,4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide;
4′-Chloro-biphenyl-4-carboxylic acid (4-{4-[1-(2-hydroxy-ethoxy)-5,6,7,8-tetrahydro-naphthalen-2-yl]-piperidin-1-yl}-butyl)-amide; and
4′-Methanesulfonylamino-biphenyl-4-carboxylic acid (4-{4-[1-(2-hydroxy-ethoxy)-5,6,7,8-tetrahydro-naphthalen-2-yl]-piperidin-1-yl}-butyl)-amide.

12. A pharmaceutical composition comprising a compound as defined in claim 1 and a pharmaceutically acceptable carrier or diluent.

13. (canceled)

14. (canceled)

15. A method for the treatment of a condition resulting from elevated circulating levels of LDL-cholesterol in a mammal in need thereof, said method comprising administering a therapeutically effective amount of a compound according to claim 1.
